(19) United States
(12) Patent Application Publication
(10) Pub. No.: US 2022/0395461 A1
(43) Pub. Date: Dec. 15, 2022

(54) SOLID PARTICLES CONTAINING SOLID PRIMARY PARTICLES THAT CONSIST ESSENTIALLY OF NATIVE CELLULOSE

(71) Applicant: Evonik Operations GmbH, Essen (DE)

(72) Inventors: Georg DUERR, Recklinghausen (DE); Sibylle SCHLEGEL-KACHEL, Bad Krozingen (DE); Johannes OEHRLEIN, Duesseldorf (DE); Juri TSCHERNJAEW, Aschaffenburg (DE); Birte BEINE, Bochum (DE); Victor BAUDRON, Frankfurt am Main (DE); Fabian-Pascal SCHMIED, Darmstadt (DE)

(73) Assignee: Evonik Operations GmbH, Essen (DE)

(21) Appl. No.: 17/775,140

(22) PCT Filed: Nov. 5, 2020
(86) PCT No.: PCT/EP2020/081125

§ 371 Date: May 6, 2022

(30) Foreign Application Priority Data
Nov. 11, 2019 (EP) 19208352.5

(51) Int. Cl. A61K 9/16; A61K 8/73; A61K 8/02; A61K 9/19; A23L 33/24

(52) U.S. Cl. CPC A61K 9/1652; A61K 8/731; A61K 8/0241; A61K 9/19; A23L 33/24; A23V 2002/00

(57) ABSTRACT

The invention relates to solid particles containing solid primary particles that consist essentially of native cellulose and optionally a binder, to the production and use thereof.

### FIELD OF THE INVENTION

The invention provides solid particles comprising solid primary particles consisting of largely native cellulose and a binder, and the production and use thereof.

### PRIOR ART

DE2921931 discloses a process for producing free-flowing products based on cellulose powder that are suitable for use in the pharmaceutical, chemical and food industries.

The object of the invention was to provide solid particles suitable for use especially in foods, cosmetics and/or pharmaceutical products, that are capable of readily absorbing active substances such as flavours, medicaments etc. and releasing them again in aqueous media.

### DESCRIPTION OF THE INVENTION

It was surprisingly found that the particles described in claim 1 can be used advantageously in foods, cosmetics and/or pharmaceutical products.

The present invention accordingly provides solid particles having an average particle size from 15 μm to 2000 μm comprising

A) solid primary particles having an average particle size from 3 μm to 20 μm, containing at least 95% by weight of native cellulose obtained from plant fibres, the percentages by weight being based on the total weight of the dry primary particles, and

B) at least one binder.

An advantage of the present invention is that the particles according to the invention are able to absorb active substances of all kinds in large amounts.

A further advantage of the present invention is that the particles according to the invention release the absorbed substances in large amounts in an aqueous medium.

A further advantage of the present invention is that the particles according to the invention release the absorbed substances very rapidly in an aqueous medium.

Another advantage of the present invention is that the particles according to the invention have excellent flowability.

A further advantage of the present invention is that the particles according to the invention are mechanically stable.

A further advantage of the present invention is that the particles according to the invention can be produced entirely on the basis of renewable raw materials.

Another advantage of the present invention is that the particles according to the invention are biodegradable.

A further advantage of the present invention is that the particles according to the invention can be processed into tablets very easily, in particular without needing to use many additives.

Another advantage of the present invention is that the tablets produced with the particles according to the invention have a high hardness.

Another advantage of the present invention is that the tablets produced with the particles according to the invention have a low mass.

The present invention thus provides solid particles having an average particle size from 30 μm to 2000 μm, preferably from 50 μm to 200 μm, more preferably from 120 μm to 180 μm, comprising

A) solid primary particles having an average particle size from 3 μm to 20 μm, preferably from 8 μm to 15 μm, more preferably from 9 μm to 12 μm, that comprise at least 95% by weight, preferably at least 97% by weight, more preferably at least 99% by weight, of native cellulose obtained from plant fibres, the percentages by weight being based on the total weight of the dry primary particles, and

B) at least one binder.

In the context of the present invention, the term “native cellulose obtained from plant fibres” is to be understood as meaning a cellulose that has undergone no chemical modification in the form of treatment with concentrated acid or base resulting in at least partial removal of the amorphous fractions of the cellulose and in particular has undergone no chemical derivatization such as hydroxypropylation, hydroxyethylation, carboxymethylation, esterification (e.g. acetylation), etherification (e.g. methylation) and quaternization, but was obtained solely from a natural substance by milling in an aqueous medium.

In the context of the present invention, the term “dry” is used to describe a cellulose that has undergone the following drying:

10 g of the cellulose is stored at 105° C. in a drying cabinet for 2 hours.

The residual moisture before drying is not more than 9%.

After cooling to room temperature in a desiccator, the sample is reweighed.

Calculation:

100%−((Weight after drying (g)×100%)/Initial weight before drying (g))=(%) Residual moisture

In the context of the present invention, the term “solid” is understood as meaning the “solid” state of aggregation at an ambient temperature at which the cosmetic formulations are used, this temperature range being in particular from 15° C. to 45° C.

All conditions such as pressure and temperature for example are, unless otherwise stated, standard conditions (25° C., 1 bar). Percentages are, unless otherwise described, expressed in percent by mass.

The average particle size was determined by laser diffraction particle size analysis in a Horiba LA 950 analyser from Retsch GmbH, Germany. The interaction of laser light with particles gives rise to light scattering patterns caused by diffraction, refraction, reflection and absorption that are characteristic of the particle size. These light scattering patterns are assigned by means of Fraunhofer theory to a particular particle size distribution, the average particle size being the d50 value for the volume-weighted particle size distribution. The analyser is able to analyse particles in the size range between 0.1-3000 μm.

The cellulose powder was measured dry. The following settings were selected on the analyser:
Compressed air: 0.40 MPa
Channel: Auto
Distribution type: Volume
Refractive index (R): Fraunhofer RT [FH RT (2000-5600i)]

The stability of the powder is determined via the abrasion during a sieving process. For the test, 5-10 g of powder was placed on a sieve having a mesh size of 63 μm and sieved on the sieve tower (10 min, 2.5 mm amplitude).

The content of cellulose in the primary particles is determined as follows:

2.5 g of comminuted sample (dry content determined in a separate sample: 100%−% LD (LD: loss on drying)) is weighed into a 150 ml beaker (W). 30 ml of 17.5% sodium hydroxide solution thermally equilibrated at 20° C. is pipetted into the sample. The mass is carefully crushed using a glass rod that is flattened at one end and allowed to stand for 30 minutes. At the end of this time, it is quickly diluted with 100 ml of RO (reverse osmosis) water, immediately stirred and filtered with suction. The G3 glass frit to be used must be first dried in a drying oven, cooled in a desiccator and weighed (B_{empty}). Rigorous care must be taken to ensure that the diluted mass is suction-filtered from the alkaline liquid as quickly as possible and that the subsequent washing is likewise carried out swiftly. The alkali is washed out with RO water added in a number of portions. Washing is continued until the washings are no longer alkaline to pH paper. 0.5% hydrochloric acid is then poured over the filter cake and the filter cake is allowed to stand for 10 minutes without suction-filtering. It is then washed again with RO water until the washings are no longer acid to pH paper. The operation takes place at 20° C. The frit is then dried to constant weight at 105° C., cooled in a desiccator and reweighed (B_{reweighed}).

$$Cellulose content ( \% ) = \frac{( {B}_{reweighed} - {B}_{empty} ) ⨯ 100 ⨯ 100}{W ⨯ ( 100 LD )}$$

The essential difference between microcrystalline celluloses and the cellulose used here is the property that the primary particles are poorly soluble in water and are present in the form of solid particles. In accordance with the invention, preference is accordingly given in particular to particles characterized in that the primary particles contained therein have a maximum solubility in water at pH 7.0, 20° C. and 1 bar from 0 g/L to 0.5 g/L, preferably from 0 g/L to 0.2 g/L, more preferably from 0 g/L to 0.08 g/L.

Preference is according to the invention given to particles characterized in that they have a bulk density of 100-300 g/L, preferably 120-270 g/L, more preferably 140-240 g/L.

The bulk density is determined in accordance with DIN 53468.

Preference is according to the invention given to particles characterized in that they have an oil absorption capacity of 1.3 g to 1.6 g limonene per g of dry particles.

The oil/water absorption capacity is determined as described in the examples.

The primary particles contained in the particles according to the invention comprise preferably native cellulose obtained from plant fibres and having a degree of crystallinity from 40 to 90%, preferably from 50 to 85%, more preferably from 60 to 80%.

The quantitative determination of the crystallinity of cellulose samples is carried out using the following peak height method, described, for example, in N. Terinte, R. Ibbett and K. C. Schuster, Lenzinger Berichte 89 (2011) 118-131:

X-ray diffraction images in the range from 5° to 45° (2Θ) are generated in reflectance mode.

The air scattering curve is determined using the pure crystalline standard NIST640c and is used as background for the X-ray diffraction patterns of the measured samples. This background is subtracted from the measured sample. The degree of crystallinity CI is calculated as the ratio of the peak height of the crystalline signal I(002) at 22° (2Θ) after subtraction of the non-crystalline contribution 1 (non-crystalline) (the signal at 18° (2Θ)) and the peak height of the crystalline peak I(002) at 22° (2Θ):

CI=((I(002)−I(non-crystalline))/I(002))*100%

The primary particles contained in the particles according to the invention comprise preferably native cellulose obtained from plant fibres and having an average degree of polymerization from 1 to 50 000, preferably 50 to 20 000, more preferably 200 to 3000.

The average degree of polymerization is determined via measurement of the relative viscosity of the cellulose dissolved in a Cuen (copper(I1)ethylenediamine) solution as described below.

1.3 g of sample is weighed into a 100 ml conical flask (WS). The dry content of the sample (LD) or the residual moisture must be determined separately.

The sample is rinsed with 25 ml of RO water, which is flushed with nitrogen before use, and the cellulose is dispersed in the water by swirling.

To this is added 25 ml of 1 M Cuen solution, which is flushed with nitrogen before use.

Nitrogen is passed into the sample solution. The conical flask is closed with the associated glass stopper. The sample is shaken until the cellulose has completely dissolved. Approx. 15 ml of this solution is transferred to an Ubbelohde viscometer having the 1c capillary. The sample solution is thermally equilibrated at 25° C.±0.1° C. A pipetting aid is used to draw the solution through the viscometer capillary until the level is above the upper glass ball. The time taken for the solution to flow from the upper to the lower mark is recorded. The process is repeated and the average value t1 of the two measured times is calculated, provided the two values do not differ by more than 1%. Otherwise, a further determination must be carried out and the average of two results differing by not more than 1% determined. Repetition of the process minus the cellulose is used to determine the blank value. This is done using capillary 1. The resulting average value t2 is the flow time of the pure Cuen solution.

Calculation of the relative viscosity:

ηrel=(t1*k1)/(t2*k2)

t1=Flow time of the sample solution (average value)

t2=Flow time of the Cuen solution (average value)

k1=Constant of the Ubbelohde viscometer, capillary 1c

k2=Constant of the Ubbelohde viscometer, capillary 1

The value for the intrinsic viscosity at the relative viscosity for the cellulose solution is taken from Table 0315.-1. in Ph. Eur. 6.3. Powdered Cellulose. The degree of polymerization is calculated as

DP=(9500*ηc)/W*(100−LD)

where

ηc: intrinsic viscosity, W: initial weight, LD: loss on drying in %.

Preference is according to the invention given to particles characterized in that the native cellulose obtained from plant fibres that is contained in the primary particles has a type I cellulose content in the crystalline fraction of greater than 95% by weight, more preferably greater than 99% by weight, based on the total crystallinity.

The different cellulose types are described, for example, in Park et al. Biotechnology for Biofuels 2010, 3:10. The cellulose type was determined by matching the X-ray diffraction patterns with the reference patterns held in the ICSD (Inorganic Crystal Structure Database). This matching was based on peak positions and intensity ratios and was done using the search function of the HighScore Plus software (manufacturer: PANalytical), version 3.0c.

Preference is according to the invention given to particles characterized in that they are spray-dried particles and have an average particle size from 120 μm to 180 μm and that the primary particles contained therein have an average particle size from 3 μm to 15 μm.

Preference is according to the invention given to particles characterized in that they contain the solid primary particles comprising native cellulose obtained from plant fibres in an amount from 60% by weight to 95% by weight, preferably 70% by weight to 90% by weight, more preferably 75% to 85% by weight.

Preference is according to the invention given to particles characterized in that the binder is selected from the group comprising, preferably consisting of, guar, alginic acid, alginate, dextrin, carbomer, maltodextrin, methyl cellulose, ethyl cellulose, gum arabic, hydroxyethyl cellulose, hydroxypropyl cellulose, hydroxypropyl methyl cellulose, hydroxyethyl methyl cellulose, carboxymethyl cellulose, cottonseed oil, povidone, ceratonia, dextrose, polydextrose, starch, gelatin, pregelatinized starch, hydrogenated vegetable oil, maltodextrin, microcrystalline cellulose, polyethylene oxide, polymethacrylates, cellulose fibres, preferably gum arabic (1-10%) carboxymethyl cellulose (8-10%) methyl cellulose (1-30%), cellulose fibres (5-15%), ethyl cellulose (1-10%) and hydroxypropyl methyl cellulose (1-10%). The values in brackets indicate preferred weight ranges based on the total particle weight of the respective binder.

Further binders may be waxes, proteins or alumina.

It is in accordance with the invention preferable that the particles contain no epoxy resin.

Preference is according to the invention given to particles characterized in that they contain the binder in an amount from 1% by weight to 30% by weight, preferably 2% by weight to 20% by weight, more preferably 5% by weight to 10% by weight.

The present invention further provides a process for producing solid particles having an average particle size from 15 μm to 2000 μm, preferably from 50 μm to 200 μm, more preferably from 120 μm to 180 μm, comprising the following process steps

A) providing solid primary particles having an average particle size from 3 μm to 20 μm, preferably from 8 μm to 15 μm, more preferably from 9 μm to 12 μm, that comprise at least 95% by weight, preferably at least 97% by weight, more preferably at least 99% by weight, of native cellulose obtained from plant fibres, the percentages by weight being based on the total weight of the dry primary particles,

B) adding liquid and a binder,

C) agglomerating the primary particles by granulating, compacting or spray drying, in particular spray drying.

The process according to the invention preferably uses those binders that are preferably contained in the particles according to the invention. The same applies to the primary particles.

### Spray Drying:

The process according to the invention for producing solid particles having an average particle size from 15 μm to 250 μm, preferably from 50 μm to 200 μm, more preferably from 120 μm to 180 μm, is preferably characterized in that liquid and preferably a binder are added in process step B) and that the agglomeration in process step C) is carried out by spray drying.

It is in accordance with the invention preferable that the binder is present in the liquid in dissolved form. The primary particles are preferably dispersed in a liquid, in particular with the aid of an intensive rotor-stator machine (e.g. IKA Ultra-Turrax). Process step B) is preferably carried out in a stirred vessel, in particular with homogenization of the liquid, optionally of the binder and of the primary particles. Preferably, the solids concentration, based on the liquid, optionally the binder and the primary particles, is 5% by weight to 30% by weight, preferably 10% by weight to 30% by weight, more preferably 15% by weight to 20% by weight.

Process step C) is preferably carried out in a spray-drying tower in which the liquid, optionally the binder and the primary particles are atomized, preferably using a two-component nozzle, a pressure nozzle or a centrifugal atomizer.

Spray drying may be carried out with a drying gas, in particular nitrogen, in cocurrent or in countercurrent. The drying gas is preferably separated from the solid particles with the aid of a cyclone.

### Granulation:

The process according to the invention for producing solid particles having an average particle size from 200 μm to 450 μm is preferably characterized in that the agglomeration in process step C) is carried out by granulation.

The process according to the invention is in this context preferably characterized in that, in process step B), the binder dissolved in the liquid is added dropwise to the primary particles or is fed in via a nozzle.

This process is further preferably characterized in that it includes the following process step:

D) drying the particles, in particular in an air-circulation oven, preferably in a temperature range from 60° C. to 140° C., preferably from 80° C. to 120° C., more preferably from 90° C. to 110° C.

A size fractionation of the solid particles to an average particle size from 200 μm to 450 μm is optionally carried out, in particular by sieving.

### Compaction:

The process according to the invention for producing solid particles having an average particle size from 200 μm to 450 μm is preferably characterized in that the agglomeration in process step C) is carried out by compaction.

In this context, it is in accordance with the invention preferable that compaction in process step C) is carried out using a roller compactor and that the process according to the invention preferably includes the following process step:

E) comminuting the compacted solid from process step C), preferably by means of a passage screen, and size fractionation of the solid particles to an average particle size from 200 μm to 2000 μm, in particular by sieving.

The primary particles and optionally the binder are preferably fed to the roller compactor by means of a stuffing screw. In this connection, it is in accordance with the invention preferable that no binder is used.

The roller compactor is preferably a Bepex L200/50 G+K (Hutt 2).

The present invention further provides the particles obtainable by the process according to the invention.

The present invention still further provides for the use of a particle according to the invention for absorption, preferably with subsequent desorption in an aqueous medium, of at least one substance selected from the group comprising flavourings, cosmetics and pharmaceutical active substances.

In this use according to the invention, preferred solid particles that are preferably previously described above are used in particular.

The examples adduced hereinbelow illustrate the present invention by way of example, without any intention of restricting the invention, the scope of application of which is apparent from the entirety of the description and the claims, to the embodiments specified in the examples.

The following figures form part of the examples:

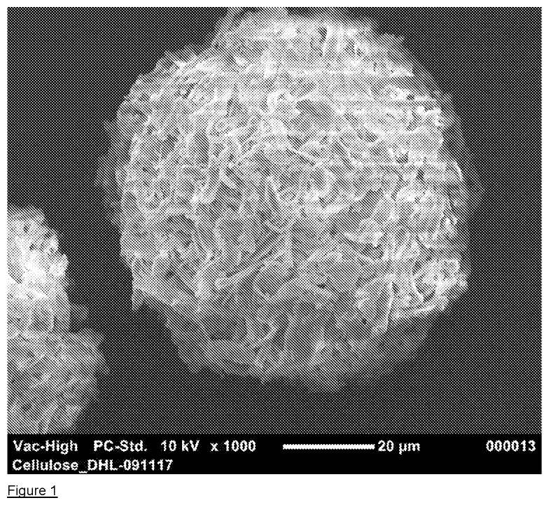

FIG. 1: Spray-dried particles comprising solid primary particles having an average particle size of 9 μm (inventive)

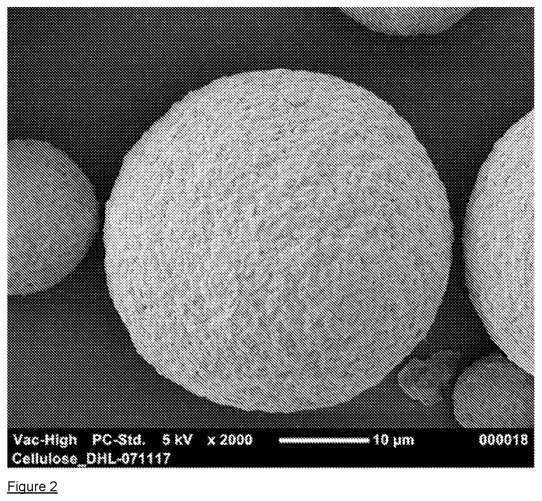

FIG. 2: Spray-dried particles comprising solid primary particles having an average particle size of 2 μm (non-inventive)

### EXAMPLES

### Example 1: Oil Absorption and Flowability

Spray-dried particles were produced as described below from commercially available native cellulose obtained from plant fibres:

### Spray Drying:

The suspension of the cellulose primary particles in water (5-25% by weight of cellulose) was prepared using a disperser and optionally mixed with a binder solution for 30 minutes using an overhead stirrer. Three different spray-drying processes were used:

a) This dispersion was then conveyed to the spray dryer (Niro Minor from GEA®-evaporation capacity: 6 kg_{H2O}/h) by means of a peristaltic pump (2.5 kg/h) and atomized using a two-component nozzle (atomizing gas: nitrogen—0.5 bar). Hot nitrogen (50 Nm³/h, Tᵢₙ=240° C., cocurrent) was used as the drying gas. The particles were separated and collected in a cyclone.

b) This dispersion was then conveyed to the spray dryer (evaporation capacity: 120 kg_{H2O}/h) by means of an eccentric screw pump (25 kg/h) and atomized using a rotary atomizer (speed of rotation: 33 Hz). Hot nitrogen (400 Nm³/h, Tᵢₙ=200° C., cocurrent) was used as the drying gas. The particles were separated and collected in a cyclone.

c) This dispersion was then conveyed to the spray dryer (evaporation capacity: 1200 kg_{H2O}/h) by means of a high-pressure pump (2000 kg/h) and atomized through a plurality of pressure nozzles (2.29 mm/40 bar). Hot nitrogen (air inflow speed: 8 m/s, Tᵢₙ=200° C., cocurrent) was used as the drying gas. The particles were dried further with hot air in a fluidized bed (Tᵢₙ=60° C.).

If binder (methyl cellulose (MC) or gum arabic (GA)) was used, an aqueous composition thereof was first prepared: Powdered binder was added at a temperature of 80° C., with stirring, to the same amount of water as is present in the cellulose dispersion to be incorporated. After 20 minutes, as soon as the binder had become finely dispersed, the same amount of water, which had a temperature of 20° C., was again added and the composition was cooled to 0-5° C. with stirring. Stirring was continued for a further 40 minutes until the binder had dissolved completely.

In the size range of the primary particles according to the invention, Tego® Feel Green and Diacel 10 and Diacel 90 were used; below the range according to the invention, finely milled Tego® Feel Green was used as the primary particles and above the claimed range Tego® C10 was used. In products 2 and 5 (Table 2), methyl cellulose was added as an additional binder. In products 6 and 7 (Table 2), gum arabic was added as an additional binder.

### Determination of Oil Absorption

Predrying the carrier material: Weigh 1.5 g of the carrier substance (cellulose) into a 100 ml screw-cap laboratory bottle and dry uncapped overnight in a vacuum drying cabinet (45° C., 20 mbar). If necessary, close the mouth with a paper towel and a rubber band to prevent loss of material when switching on the vacuum.

Loading the carrier material: After this, load the dried samples with the 3 g of oil (limonene) and mix well with a spatula. Ensure as far as possible that too much mixture does not stick to the spatula. Screw the cap on the bottle and allow to stand for approx. 3 hours.

Centrifugation: Fold 5 round filters (Ø5.5 cm) into a funnel shape and insert into a 50 ml Falcon tube. Weigh 3 g of the cellulose-oil mixture into the Falcon tube, making sure that the mixture is unable to bypass the filter by running down the side and that it does not stick to the side of the Falcon tube. Centrifuge the filled Falcon tube (Hettich Rotina 380R centrifuge, rotor radius: 14.8 cm/4300 rpm/duration: 6 min).

Weighing the filter cake: Determine the empty weight of a suitable glass dish (as small as possible, since oversized dishes result in inaccurate weight measurements on the analytical balance). Place the entire filter cake in the glass bowl, break it up a little with the spatula and weigh it.

Drying the filter cake: After this, dry the filled glass dish in the vacuum drying cabinet for at least 12 hours (45° C., 20 mbar) and then reweigh (again covering the glass dish with a paper towel and a rubber band). From the difference in weight before and after drying, determine the loading of the carrier with oil prior to drying.

### Determination of Angle of Repose

The angle of repose was measured in accordance with ISO 4324.

### Determination of Flowability

The flowability of the carrier substances was determined with the aid of a series of glass funnels having different outlet openings. For the test, the funnels are held in place with a holder above a collecting vessel. To cover the funnel opening, a playing card is clamped between the funnel and the vessel. The funnel is filled with the carrier substance to a height two cm below the upper edge of the funnel. The card is then removed and the powder runout is assessed on the basis of the following scale.

TABLE 1
Properties of cellulose primary particles.

| | Runout from funnel | |
|---|---|---|
| Rating | of d [mm]* | Flowability |
| 1 | 2.5 | very good |
| 2 | 5.0 | |
| 3 | 8.0 | |
| 4 | 12.0 | |
| 5 | 18.0 | |
| 6 | 24.0 | poor |
| 7 | — | no runout from 6 |

| | | | | Stability of | |
|---|---|---|---|---|---|
| | d50 of the | Flowability | Oil absorption | the primary | |
| | primary | of the | capacity | particles; | Bulk |
| | particles | primary | g oil/g primary | fines fraction/ | density |
| Primary particles | in μm | particles | particles | abrasion in % | in g/L |
| 1: Tego ® Feel | 8.75 | 7 | 1.2 | 93 | 183 |
| Green | | | | | |
| 2: Diacel 10 | 9.0 | 7 | 1.1 | — | 200 |
| 3: Diacel 90 | 38 | 7 | — | — | 224 |
| 4: Milled Tego ® | 3 | / | / | / | / |
| Feel Green | | (aqueous | (aqueous | (aqueous | (aqueous |
| | | suspension) | suspension) | suspension) | suspension) |
| 5: Tego ® C10 | 44 | 7 | 1.2 | — | 252 |
| 6: Microcrystalline | 150 | 7 | 0.4 | 49 | 345 |
| cellulose (MCC) | | | | | |

*Sample runs out smoothly with a single tap; if this occurs only after 2-4 taps, a rating of +0.5 is given.
“—” not determined;
“/” measurement not possible

The primary particles 1-5 (Tab. 1) consist of at least 95% by weight of native cellulose obtained from plant fibres.

TABLE 2a
Results for the production of cellulose particles.

| | | d50 of | Flowability of | Oil-absorption | Bulk | Angle of |
|---|---|---|---|---|---|---|
| | Spray-drying | particles | spray-dried | capacity | density | repose |
| Spray-dried product | process | in μm | products | g oil/g particles | in g/L | in ° |
| 1: Diacel 10 | a) | 50 | | 1.31 | 242 | 40 |
| 2*: Diacel 10 with 10% | a) | 66 | | 1.2 | 253 | 30 |
| by weight MC | | | | | | |
| 3: Milled Tego ® Feel | a) | 18 | | 0.7 | 596 | — |
| Green | | | | | | |
| 4: Tego ® C10 | no particles formed | | | | | |
| 5*: Tego ® Feel Green | a) | 62 | 4 | 1.37 | 235 | — |
| with 10% by weight | | | | | | |
| MC | | | | | | |
| 6*: Diacel 10/Diacel 90 | b) | 187 | 1 | 1.0 | 280 | 30 |
| (90:10% by weight) + | | | | | | |
| 10% by weight GA | | | | | | |
| 7*: Diacel 10 + 10% | b) | 182 | 1 | 1.0 | 300 | 30 |
| by weight GA | | | | | | |

“—” not determined;
“/” measurement not possible;
“*” inventive

TABLE 2b
Results for the production of cellulose particles.

| | | | Flowability | Oil absorption | | |
|---|---|---|---|---|---|---|
| | | d50 of | of | capacity | Bulk | Angle of |
| Spray-dried | Spray-drying | particles | spray-dried | g oil/g | density | repose |
| product | process | in μm | products | particles | in g/L | in ° |
| 8: Emcocel | — | 216 | 2 | 0.8 | 200-370 | No flow |
| LP200 (MCC) | | | | | | |
| 9: Emcocell 90M | — | 112 | 5 | 0.9 | 250-370 | 37 |
| (MCC) | | | | | | |
| 10: Vivapur 200 | — | 218 | 5 | 0.7 | 310-370 | 34 |
| (MCC) | | | | | | |
| 11: Vivapur 12 | — | 140 | 5 | 0.8 | 300-360 | 37 |
| (MCC) | | | | | | |
| 12:Vitacel CS | — | 308 | 2 | 0.5 | 370 | — |
| 250G (cellulose) | | | | | | |
| 13: Sanacel | — | 59 | 7 | 1.1 | — | 53 |
| pharma 150 | | | | | | |
| (cellulose) | | | | | | |

“—” not determined;
“/” measurement not poossible;
“*” inventive

It is immediately evident from the results listed in Table 2 that the spray drying of primary particles in the size range according to the invention results in particles having increased oil absorption capacity. If the primary particles are too small, the oil absorption capacity is low; if they are too large, no flowable particles are obtained.

The inclusion of binders such as methyl cellulose or gum arabic increases the mechanical stability of the particles, characterized by reduced fines formation.

### Example 2: Absorption and Desorption of Pharmaceutical Active Substances

4-[5-(4-Methylphenyl)-3-(trifluoromethyl)-pyrazol-1-yl]benzenesulfonamide (celecoxib, Aarti Drugs Ltd., Mumbai, India) was loaded on various cellulose preparations.

The active substance celecoxib was incorporated in a mixture of different components consisting of Miglyol® 812, Tween® 80, Gelucire® 44/14 and D-α-tocopherol polyethylene glycol 1000 succinate (d-TPGS).

The cellulose, MCC and silica preparations are loaded with the latter oily formulation, which contains celecoxib.

Desorption was investigated with 25 mg or an equivalent amount of celecoxib in a USP Apparatus II (Pharma Test PWTS 1210) in 500 ml of 0.1 N HCl at 100 rpm and 37±0.5° C. (HPLC (Agilent 1260 Infinity), HPLC pump (G1311B), autosampler (G1329B), column oven (G1316A) and UV detector (G1314C), from Agilent Technologies (USA), Knauer Nucleosil 100-7 C18 (125×4.6 mm, 7 μm) column, 40° C., mobile phase acetonitrile:water:trimethylamine mixture (300:300:0.9 v/v), adjusted to pH 3.00 with phosphoric acid, flow rate 1.8 ml/min, injected volume 5 μl, celecoxib measured at 254 nm, limit of quantitation 0.05 μg/ml, run time 7 min).

The products 1, 2, 3 and 4 (Table 3) were produced by spray-drying process a), and products 9 and 10 (Table 3) by spray-drying process c), of example 1. Product 5 (Table 3) was produced using the process of the invention, by process step C) granulation in an intensive mixer (Eirich model ELS Eco). This was done by charging the mixer bowl with Tego® 010 cellulose fibres and adding the starch adhesive solution via a nozzle. Mixing was then continued for a certain period. The granules were dried overnight in an air-circulation oven at 100° C. and finally graded through sieves. The target fraction was initially set at 200-410 μm. The starch adhesive solution was prepared by adding 125 g of cornstarch to 500 ml of hot water (90-95° C.) with vigorous stirring. The temperature was maintained for 15 min to achieve gelatinization of the starch.

TABLE 3a
Results for the production of cellulose particles.

| | | | | | | | Release in |
|---|---|---|---|---|---|---|---|
| | Spray- | d50 of | Loading | Release | Release | Release | g_{formulation}/ |
| | drying | particles | g_{formulation}/ | in % after | in % after | in % after | g_{carrier} |
| | process | in μm | g_{carrier} | 5 min | 10 min | 15 min | after 5 min |
| 1: Diacel 10 | a) | 14 | 1.15 | 82 | 91 | 95 | 0.94 |
| 2*: Diacel 10 + | a) | 16 | 1.23 | 80 | 88 | 95 | 0.98 |
| 9% by weight | | | | | | | |
| MC | | | | | | | |
| 3*: Diacel 10 + | a) | 18 | 1.26 | 88 | 98 | 100 | 1.11 |
| 17% by | | | | | | | |
| weight MC | | | | | | | |
| 4*: Diacel 10 + | a) | 19 | 1.26 | 80 | 93 | 96 | 1.01 |
| 23% by weight | | | | | | | |
| MC | | | | | | | |
| 5*: Tego ® Feel | Granulation | 300 | 1.29 | 83 | 89 | 90 | 1.07 |
| Green + 6% | | | | | | | |
| by weight | | | | | | | |
| starch | | | | | | | |
| adhesive | | | | | | | |
| 6: Avicel PH- | — | 50 | 0.90 | 83 | 86 | 89 | 0.75 |
| 101 (MCC) | | | | | | | |
| 7: Aeroperl ® | — | 30 | 1.40 | 23 | 30 | 32 | 0.32 |
| 300 Pharma | | | | | | | |
| (silica) | | | | | | | |

“*” inventive

TABLE 3b
Results for the production of cellulose particles.

| | | | | | | | Release in |
|---|---|---|---|---|---|---|---|
| | Spray- | d50 of | Loading | Release | Release | Release | g_{formulation}/ |
| | drying | particles | g_{formulation}/ | in % after | in % after | in % after | g_{carrier} |
| | process | in μm | g_{carrier} | 5 min | 10 min | 15 min | after 5 min |
| 8: Syloid ® | — | 50 | 1.44 | 17 | 21 | 22 | 0.24 |
| XDP 3050 | | | | | | | |
| (silica) | | | | | | | |
| 9* Diacel 10/ | c) | 187 | 1.15 | 93 | 99 | 99 | 1.07 |
| Diacel 90 | | | | | | | |
| (90:10% by | | | | | | | |
| weight) + 10% | | | | | | | |
| by weight GA | | | | | | | |
| 10* Diacel | c) | 182 | 1.14 | 92 | 98 | 98 | 1.05 |
| 10 + 10% by | | | | | | | |
| weight GA | | | | | | | |
| 11: Emcocel | — | 216 | 0.83 | 85 | 93 | 95 | 0.71 |
| LP200 (MCC) | | | | | | | |
| 12: Emcocell | — | 112 | 0.72 | 85 | 95 | 96 | 0.61 |
| 90M (MCC) | | | | | | | |
| 13: Vivapur | — | 218 | 0.66 | 84 | 92 | 92 | 0.55 |
| 200 (MCC) | | | | | | | |
| 14: Vivapur 12 | — | 140 | 0.81 | 85 | 92 | 94 | 0.69 |
| (MCC) | | | | | | | |
| 15: Vitacel CS | — | 308 | 0.39 | 86 | 93 | 94 | 0.34 |
| 250G | | | | | | | |
| (cellulose) | | | | | | | |
| 16: Sanacel | — | 59 | 0.98 | 85 | 94 | 95 | 0.83 |
| pharma 150 | | | | | | | |
| (cellulose) | | | | | | | |

“*” inventive

It is immediately evident from the results listed in the table that the particles according to the invention achieve very high loading rates and that these very high loading rates in turn result in more rapid release of pharmaceutical active substances than is the case for conventional particles (g of formulation per g of carrier released after 5 min). Compared with silicon dioxide-based absorbents (see Table 3), the particles according to the invention likewise release the active substance more rapidly and to a greater degree.

In addition, the particles according to the invention have better flowability than the conventional microcrystalline cellulose (Avicel PH-101), show flowability comparable to that of silicon dioxide-based absorbents and are also suitable for the production of tablets and capsule fillings.

### Example 3: Tableting

The particles according to the invention were further processed into tablets on their own and in combination with other components. This process step was carried out using the EP-1 tablet press (eccentric press) from Erweka GmbH (Heusenstamm, Germany). The thickness, diameter and hardness of the various tablets was determined using the TBH 125 tablet hardness tester, likewise from Erweka GmbH (Heusenstamm, Germany). For the determination of the tablet parameters mentioned, 2 tablets (n=2) were in each case analysed for each different composition.

For the examples in Table 4, a tablet formulation was produced from the following constituents: lactose monohydrate (46.2%), talc (3.00%), silica (colloidal) (0.5%), various particle types (30.0%), maize starch (5.0%), magnesium stearate (0.3%) and celecoxib (15.0%).

TABLE 4
Results for the tableting of cellulose particles.

| | Spray-drying | Thickness** | Diameter** | Hardness** |
|---|---|---|---|---|
| | process | [mm] | [mm] | [N] |
| 1*: Diacel | a) | 4.82 ± 0.115 | 10.070 ± 0.010 | 46.0 ± 1.0 |
| 10 + 10% by | | | | |
| weight MC | | | | |
| 2*: Diacel 10/Diacel 90 | c) | 3.63 ± 0.007 | 10.02 ± 2.828 | 40 ± 0.014 |
| (90:10% by weight) + | | | | |
| 10% by weight GA | | | | |
| 3*: Diacel | c) | 3.5 ± 0.148 | 9.99 ± 0 | 48 ± 0 |
| 10 + 10% by | | | | |
| weight GA | | | | |
| 4: Emcocel | — | 3.44 ± 0.007 | 9.975 ± 2.121 | 44.5 ± 0.007 |
| LP200 (MCC) | | | | |
| 5: Vivapur | — | 3.44 ± 0.078 | 9.98 ± 4.95 | 38.5 ± 0 |
| 200 (MCC) | | | | |
| 6: Sanacel | — | 3.55 ± 0.007 | 10.01 ± 1.414 | 56 ± 0.028 |
| Pharma 150 | | | | |

**n = 2 ± SD
“*” inventive

The particles according to the invention can be further processed into tablets using the mentioned eccentric press. For comparability of the materials with one other, tablets having a thickness in the range of approx. 4.3-5.0 mm and a diameter of approx. 10.0-10.1 mm were produced.

The hardness of the tablets obtained (see Table 4) is for the listed materials in the range of approx. 40-50 N. This hardness thus determined means that the tablets based on the particles according to the invention are likewise suitable for further processing (for example coating). The hardness of the products according to the invention is comparable to the MCC products.

1. Solid particles having an average particle size from 15 μm to 2000 μm, comprising:
solid primary particles,
wherein the solid primary particles have an average particle size from 3 μm to 20 μm with at least 95% by weight of native cellulose obtained from plant fibres; and
at least one binder.

2. The particles according to claim 1, which are spray-dried particles, granulated particles, or compacted particles.

3. The particles according to claim 1, which are spray-dried particles having an average particle size from 15 μm to 250 μm.

4. The particles according to claim 1, which are granulated particles having an average particle size from 200 μm to 450 μm.

5. The particles according to claim 1, which are compacted particles having an average particle size from 200 μm to 2000 μm.

6. The particles according to claim 1, wherein the solid primary particles contained therein have a maximum solubility in water at pH 7.0, 20° C. and 1 bar from 0 g/L to 0.5 g/L.

7. The particles according to claim 1, which have a bulk density of 100-300 g/L.

8. The particles according to claim 1, wherein the native cellulose obtained from plant fibres has a crystallinity factor from 40 to 90%.

9. The particles according to claim 1, wherein the native cellulose obtained from plant fibres has an average degree of polymerization of 1 to 50 000.

10. The particles according to claim 1, wherein the native cellulose obtained from plant fibres has a type 1 cellulose content in the crystalline fraction of greater than 95% by weight based on the total crystallinity.

11. The particles according to claim 1, wherein the particles are spray-dried particles having an average particle size from 120 μm to 180 μm, and
wherein the primary particles contained therein have an average particle size from 3 μm to 15 μm.

12. The particles according to claim 1, wherein the binder is at least one selected from the group consisting of guar, alginic acid, alginate, dextrin, carbomer, maltodextrin, methyl cellulose, ethyl cellulose, gum arabic, hydroxyethyl cellulose, hydroxypropyl cellulose, hydroxypropyl methyl cellulose, hydroxyethyl methyl cellulose, carboxymethyl cellulose, cottonseed oil, povidone, ceratonia, dextrose, polydextrose, starch, gelatin, pregelatinized starch, hydrogenated vegetable oil, maltodextrin, microcrystalline cellulose, polyethylene oxide, polymethacrylates, and cellulose fibres.

13. A method for producing solid particles having an average particle size from 15 μm to 2000 μm, comprising:
adding a liquid and a binder to solid primary particles having an average particle size from 3 μm to 20 μm that comprise at least 95% by weight of native cellulose obtained from plant fibres; and
agglomerating the primary particles by granulating, compacting, or spray drying.

14. The method according to claim 13,
wherein the solid particles have an average particle size from 200 μm to 450 μm, and
wherein the agglomeration is carried out by granulation.

15. The method according to claim 13, wherein the binder dissolved in the liquid is added dropwise to the primary particles or is fed in via a nozzle.

16. The method according to claim 14, further comprising:
drying the particles in a temperature range from 60° C. to 140° C.

17. The method according to claim 13,
wherein the solid particles have an average particle size from 200 μm to 2000 μm, and
wherein the agglomeration is carried out by compaction.

18. The method according to claim 17, wherein the compaction is carried out using a roller compactor.

19. Particles obtained by the method according to claim 13.

20. A method of absorption in an aqueous medium, comprising:
contacting the particle according to claim 1, with at least one substance selected from the group consisting of flavourings, cosmetics, and pharmaceutical active substances.
